(19) United States
(12) Patent Application Publication
(10) Pub. No.: US 2008/0206369 A1
(43) Pub. Date: Aug. 28, 2008

(54) Nutritional supplement preparation with piperine

(75) Inventor: Gerhard Schmitt, Bensheim (DE)

Correspondence Address: AKERMAN SENTERFITT, 801 PENNSYLVANIA AVENUE N.W., SUITE 600, WASHINGTON, DC, 20004, US

(73) Assignee: PM-International AG, Luxembourg (LU)

(21) Appl. No.: 12/081,461

(22) Filed: Apr. 16, 2008

Related U.S. Application Data
(63) Continuation of application No. 11/360,618, filed on Feb. 24, 2006, now abandoned.

(30) Foreign Application Priority Data
Feb. 24, 2005 (EP) 05 004 035.1

(51) Int. Cl. A61K 36/67; A61K 47/02; A61K 31/4545

(52) U.S. Cl. 424/734; 514/769; 514/316

(57) ABSTRACT

A nutritional supplement preparation is provided that includes minerals and trace elements such as vitamin D, calcium, magnesium, selenium, copper, zinc and chromium, as well as at least one catalyst and/or active ingredient, preferably in powder form, which increases and/or improves and/or accelerates the absorption of the minerals and/or trace elements.

### CROSS REFERENCE TO RELATED APPLICATIONS

This application is a continuation application of U.S. patent application Ser. No. 11/360,618, filed Feb. 24, 2006, and claims priority from European Patent Application No. 05 004 035.1, filed Feb. 24, 2005, the entire contents of which in incorporated by reference.

### BACKGROUND OF THE INVENTION

1. Field of the Invention

The invention relates to nutritional supplement preparation containing minerals and trace elements (active ingredients) including vitamin D, calcium, magnesium, selenium, copper, zinc and chromium.

Magnesium influences conduction processes in the central and peripheral nervous system. Selenium is an anti-oxidative protective substance and is a building block in many enzyme systems. Chromium supports the glucose tolerance factor and therewith increases the response behavior to insulin. Chromium consequently fulfills an important function in sugar metabolism.

2. Description of the Related Art

A disadvantage with known nutritional supplement preparations is how long it takes for the minerals and trace elements to be absorbed (assimilated) after intake. Furthermore, minerals and trace elements contained together may mutually interfere with one another due to insufficient harmonization. For example, the absorption channels for a mineral or a trace element can be occupied by one or more other minerals or trace elements. The transport of individual active ingredients to the site of action can also be impeded by other active ingredients.

In addition, several active ingredients may not be compatible with one another. For example, calcium and magnesium mutually interfere with each other if their relative proportion and/or their molecular integration are not correct. Incorrect relative proportions and/or molecular integration can, for example, lead to complex formation.

### SUMMARY OF INVENTION

The present invention provides a nutritional supplement preparation with improved absorption properties for minerals and trace elements. Furthermore, the various minerals and trace elements are brought into harmony with one another so that they do not disturb one another in their absorption and in their transport to the sites of activity.

The present invention provides a nutritional supplement preparation containing the minerals and trace elements including vitamin D, calcium, magnesium, selenium, copper, zinc, and chromium, wherein the preparation has at least one catalyst and/or active substance, preferably in powder form, which increases and/or improves and/or accelerates the absorption of minerals and/or trace element. Advantageous refinements become apparent from the various embodiments

In accordance with the above, the nutritional supplement preparation contains at least one catalyst and/or active agent (also designated as REC-Resorption-Enhancing Catalyst) in addition to the minerals and trace elements that include vitamin D, calcium, magnesium, selenium, copper, zinc and chromium, preferably in powder form, which increases and/or improves and/or accelerates the absorption of the minerals and/or trace elements.

The present invention provides improved absorption properties (including improved transport of the active ingredients to their site of action) of the nutritional supplement preparation. In this way, the minerals and/or trace elements are absorbed more rapidly into the body and can unfold their actions with less of a time interval from intake. The catalyst and/or active ingredient can also improve the effectiveness of the minerals and/or trace elements in the body. According to various embodiments, the nutritional supplement preparation counteracts osteoporosis and hyperacidity.

### DETAILED DESCRIPTION OF THE INVENTION

After an advantageous refinement, the catalyst and/or active ingredient includes a black pepper extract or it consists of black pepper extract. According to one embodiment, an effective catalyst and/or active ingredient may be extracted from black pepper (Piper nigrum).

In accordance with a preferred embodiment, the catalyst and/or active substance includes piperine (1-piperoylpiperidine C₁₇H₁₉NO₃ is a chemical variant), especially effective as an alkaloid, or it consists of it. A piperine, which is effective as a catalyst and/or active ingredient, can be obtained on the basis of pepper (Piper nigrum) as an initial substance.

According to one embodiment of the invention, it is advantageous if the proportion of piperine in the catalyst and/or the active ingredient lies in the range of 90 to 100 percent, especially in the range of 92 to 100 percent, preferably at least at 95 percent.

According to one embodiment, the catalyst and/or active substance remains substantially completely or at least as far as possible in its molecular structure as it exists in the black pepper. Moreover, in a preferred embodiment, the black pepper is a substance extracted from the plant black pepper rather than a synthetic chemical product. A special grinding may provide (at least to the greatest extent possible) the maintenance of the natural molecular structure.

According to some embodiments, it has proven to be particularly advantageous if the nutritional supplement preparation contains magnesium carbonate hydroxide. The use of magnesium carbonate hydroxide leads to an improved capacity to absorb magnesium, as well as to an increase in its effectiveness. However, many of the magnesium compounds offered on the market are generally hard to absorb. Previously, the use of magnesium carbonate was typical.

More appropriately, the nutritional supplement preparation contains according to the execution variant beta arotene (provitamin A) and/or potassium and/or sodium and/or iron.

Iron is an additive that, among other things, has proven advantageous with strong hyperacidity. Especially high performance athletes and women should therefore use a nutritional supplement preparation with an iron component in order to assure the synthesis of hemoglobin.

A preferred refinement of the nutritional supplement preparation provides that the potassium component in the preparation lies between 0.15 and 35% by weight, preferably between 0.5 and 10% by weight, especially at 0.8% by weight, and/or that the calcium component in the preparation lies between 1.6 and 17% by weight, preferably between 2.5 and 10% by weight, especially at 4.5% by weight, and/or that the magnesium component lies between 0.8 and 5.0% by weight, preferably between 2 and 3% by weight, especially at 2.2% by weight, and/or that the zinc component in the preparation lies between 0.015 and 0.2% by weight, preferably between 0.05 and 0.1% by weight, especially at 0.08% by weight, and/or that the copper component in the preparation lies between 0.0015 and 0.017% by weight, preferably between 0.005 and 0.015% by weight, especially at 0.011% by weight, and/or that the chromium component in the preparation lies between 0.00015 and 0.0017 %, preferably between 0.0002 and 0.00008% by weight, especially at 0.00028%, and/or that the selenium component in the preparation lies between 0.00015 and 0.0012% by weight, preferably between 0.0002 and 0.0006% by weight, especially at 0.00028% by weight, and/or that the iron component in the preparation lies at almost 0% by weight, or between 0.015 and 0.09% by weight, preferably between 0.03 and 0.09% by weight, especially at 0.08% by weight, and/or that the vitamin D component in the preparation lies between 0.00001 and 0.00008% by weight, preferably between 0.00002 and 0.00006% by weight, especially at 0.00004% by weight, and/or that the proportion in catalysts and/or active substances for improving absorption, especially the piperine component in the preparation lies between 0.15 and 1.7% by weight, preferably between 0.4 and 1.2% by weight, especially at 0.8% by weight.

Appropriate and preferred embodiments provide that the nutritional supplement preparation exists in a powder form and/or is formed and designed for manufacture as an aqueous solution and/or formed and designed for manufacture of a beverage, especially a mineral based beverage. The preparation is especially simple, flexible and quick to take as a beverage, owing to which a rapid action is assured. A beverage also makes possible a problem-free and comfortable regular use without which the desired effects cannot as a rule be attained in humans.

Furthermore, a nutritional supplement preparation set has proven appropriate that includes as a basic substance a first nutritional supplement preparation according to the invention which contains no iron, and contains a second nutritional supplement preparation according to the invention which contains iron as an additive. The basic substance and/or additive can moreover be suited for individual use. In particular, the basic substance and additive can be mixed in any desired proportion to set a desired iron proportion. The significance of iron for specified human groups, especially athletes and/or women, is explained above. The nutritional supplement preparation set makes it possible for the respective user to adjust the iron proportion corresponding to his/her individual requirements by corresponding mixing of the basic substance and additive to produce an optimally composed preparation.

The characteristic values of an embodiment of the nutritional preparation of the invention will be presented below.

Proceeding from a nutritional supplement preparation portion of 6 g (grams), which corresponds to the daily amount to be taken, the following table provides typical amount values of the individual minerals or trace elements within this proportion. That means that the daily portion of 6 g contains 50 mg (milligrams) of potassium, 270 mg of calcium, etc. Additional unit used: μg (micrograms). Furthermore, the limits within which the respective substance can be varied are indicated. Additional substances are contained in addition to the indicated substances that, for example, serve the aroma and/or the solubility of the nutritional supplement preparation. In this way, good solubility and acceptable taste of the nutritional supplement preparation is attained.

| | Amount in a 6 gram | | |
|---|---|---|---|
| Mineral or trace element | portion | | Limiting value |
| Potassium | 50 | mg | 10 mg-2,000 mg |
| Calcium | 270 | mg | 100 mg-1,000 mg |
| Magnesium | 134 | mg | 50 mg-300 mg |
| Zinc | 5 | mg | 1 mg-11 mg |
| Copper | 670 | μg | 100 μg-1 mg |
| Chromium | 17 | μg | 10 μg-100 μg |
| Selenium | 17 | μg | 10 μg-70 μg |
| Iron | 5 | mg | 1 mg-5 mg |
| Vitamin D | 2.5 | μg | 1 μg-5 μg |

Furthermore the amount of piperine per 6 gram portion can typically lie from 0.1 mg to 10 mg, preferably from 0.3 mg to 2 mg, especially at 0.5 mg.

In a nutritional supplement preparation set, the basic substance corresponds to the above composition except for the iron value, which lies at 0 grams. In contrast the additive corresponds to the above composition including the iron value. The user can then adjust the individually desired iron value by appropriate mixture of basic substance and additive.

While the preferred forms of the invention have been disclosed, it will be apparent to those skilled in the art that various changes and modifications may be made that will achieve some of the advantages of the invention without departing from the spirit and scope of the invention. It will be apparent to those reasonably skilled in the art that other components performing the same function may be suitably substituted. Therefore, the scope of the invention is to be determined solely by the appended claims.

What is claimed is:

1. A method for providing a first nutritional supplement preparation as a basic substance and a second nutritional supplement preparation as an additive, wherein the first nutritional supplement comprises:
minerals and trace elements including vitamin D, calcium, magnesium, selenium, copper, zinc, and chromium, wherein the first nutritional supplement preparation comprises:
providing a catalyst, an active substance, or a combination of the catalyst and the active substance that accelerate absorption of the minerals or the trace element; wherein the first preparation does not include iron; wherein the second nutritional supplement comprises:
mineral and trace elements including vitamin D, calcium, magnesium, selenium, copper, zinc and chromium, wherein the second nutritional supplement preparation comprises:
providing a catalyst, an active substance, or a combination of the catalyst and the active substance that accelerate absorption of the minerals or the trace elements; wherein the second preparation contains iron, whereby the basic substance, the additive, or a combination of the basic substance and the additive is suited for individual use; and whereby the basic substance and additive can be mixed in any desired proportion to set a desired iron proportion.

2. The method according to claim 1, wherein the first and/or second nutritional supplement preparation, the catalyst, the active substance, or the combination of the catalyst and the active substance are in powder form.

3. The method according to claim 1, wherein in the first and/or second nutritional supplement preparation the catalyst, the active substance, or the combination of the catalyst and the active substance include a black pepper extract.

4. The method according to claim 1, wherein in the first and/or second nutritional supplement preparation the catalyst, the active substance, or the combination of the catalyst and the active substance include at least piperine (chemical designation 1-piperoylpiperidine C₁₇H₁₉NO₃), as an alkaloid, wherein the piperine is extracted from black pepper.

5. The method according to claim 4, wherein the piperine component is present in the catalyst or active substance in a range from 90% by weight to 100% by weight.

6. The method according to claim 4, wherein the piperine component is present in the catalyst or active substance in a range from 92 to 100% by weight.

7. The method according to claim 4, wherein the piperine component is present in the catalyst or active substance at least 95% by weight.

8. The method according to claim 3, wherein in the first and/or second nutritional supplement preparation the catalyst, the active substance, or the combination of the catalyst and the active substance remain substantially in a molecular structure as present in the black pepper.

9. The method according to claim 1, further comprising providing magnesium carbonate hydroxide in the first and/or second nutritional supplement preparation.

10. The method according to claim 1, further comprising providing beta carotene (provitamin A), potassium, sodium, iron, or any combination thereof in the first and/or second nutritional supplement preparation.

11. The method according to claim 10, further comprising, in the first and/or second nutritional supplement preparation:
providing the potassium in a concentration that lies between 0.15 and 35% by weight, between 0.5 and 10% by weight, or at 8% by weight;
providing the calcium in a concentration that lies between 1.6 and 17% by weight, between 2.5 and 10% by weight, or at 4.5% by weight;
providing the magnesium in a concentration that lies between 0.8 and 5.0% by weight, between 2 and 3% by weight, or at 2.2% by weight;
providing the zinc in a concentration that lies between 0.015 and 0.2% by weight, between 0.05 and 0.1% by weight, or at 0.08% by weight;
providing the copper in a concentration that lies between 0.0015 and 0.017% by weight, between 0.005 and 0.015% by weight, or at 0.011% by weight;
providing the chromium in a concentration that lies between 0.00015 and 0.0017%, between 0.0002 and 0.00008% by weight, or at 0.00028% by weight;
providing the selenium in a concentration that lies between 0.000158 and 0.0012% by weight, between 0.0002 and 0.0006% by weight, or at 0.00028% by weight;
providing the iron in a concentration that lies at 0% by weight, between 0.015 and 0.09% by weight, between 0.03 and 0.09% by weight, or 0.08% by weight;
providing the vitamin D in a concentration that lies between 0.00001 and 0.00008% by weight, between 0.00002 and 0.00006% by weight, or at 0.00004% by weight; and
providing the piperine in a concentration that lies between 0.0015 and 0.17% by weight, between 0.005 and 0.034% by weight, or at 0.008% by weight.

12. The method according to claim 1, wherein in the first and/or second nutritional supplement preparation the catalyst, the active substance, or the combination of the catalyst and the active substance is in a powder form and is constructed and determined for manufacture of an aqueous solution, a beverage, or a mineral based beverage.

13. A nutritional supplement preparation set comprising:
as a basic substance, a first nutritional supplement preparation containing mineral materials and trace elements including vitamin D, calcium, magnesium, selenium, copper, zinc and chromium; and
at least one catalyst and/or active ingredient which at least one of increases, improves or accelerates the resorption of the mineral materials and/or the trace elements;
wherein the first preparation contains no iron; and
as an additive, a second nutritional supplement preparation containing minerals materials and trace elements including vitamin D, calcium, magnesium, selenium, copper, zinc, and chromium; and
at least one of a catalyst, and/or active ingredient which at least one of increases, improves, or accelerates the resorption of the mineral materials and/or trace elements;
wherein the second preparation contains iron;
wherein the basic substance an/or additive are suitable for independent use; and wherein the basic substance and the additive can be mixed in any ratio for adjustment of a required iron proportion.

14. The nutritional supplement preparation set according to claim 13, wherein in each of the first preparation and/or the second preparation, the at least one catalyst and/or active ingredient is present in powder form.

15. The nutritional supplement preparation set according to claim 13, wherein in each of the first and/or the second preparation, the catalyst and/or active ingredient comprises at least a black pepper extract.

16. The nutritional supplement preparation set according to claim 13, wherein in each of the first and/or the second preparation, the catalyst and/or active ingredient comprises at least piperine (chemical designation 1-piperoylpiperidine C₁₇H₁₉NO₃), in as an alkaloid, wherein the piperine is recovered from black pepper.

17. The nutritional supplement preparation set according to claim 16, wherein each of the first and/or the second preparation, a piperine proportion in the catalyst and/or active ingredient lies in a range of 90 wt. % to 100 wt. %, and in particular in a range of 92 to 100 wt. %, and is preferably at least 95 wt. %.

18. The nutritional supplement preparation set according to claim 13, wherein each of the first and/or the second preparation, the catalyst and/or active ingredient substantially remains left in its molecular structure, as exists in black pepper.

19. The nutritional supplement preparation set according to claim 13, wherein in each of the first and/or second preparation, the first and/or the second preparation contains magnesium hydroxide carbonate.

20. The nutritional supplement preparation set according to claim 13, wherein each of the first/and or the second preparation, the first and/or the second preparation contains at least one of beta-carotene (provitamin A), potassium, or sodium.

21. The nutritional supplement preparation set according to claim 1, wherein in each of the first and/or the second preparation, a potassium proportion in the first and/or the second preparation lies between 0.15 and 35 wt. %, preferably between 0.5 and 10 wt. %, in particular at 0.8 wt. %, and/or a calcium proportion in the first and/or the second preparation lies between 1.6 and 17 wt. %, preferably between 2.5 and 10 wt. %, in particular at 4.5 wt. %, and/or a magnesium proportion in the first and/or the second preparation lies between 0.8 and 5.0 wt. %, preferably between 2 and 3 wt. %, in particular at 2.2 wt. %, and/or a zinc proportion in the first and/or the second preparation lies between 0.015 and 0.2 wt. %, preferably between 0.05 and 0.1 wt. %, in particular at 0.08 wt. %, and/or a copper proportion in the first and/or the second preparation lies between 0.0015 and 0.017 wt. %, preferably between 0.005 and 0.015 wt. %, in particular at 0.011 wt. %, and/or a chromium proportion in the first and/or the second preparation lies between 0.00015 and 0.0017 wt. %, preferably between 0.0002 and 0.0008 wt. %, in particular at 0.00028 wt. %, and/or a selenium proportion in the first and/or the second preparation lies between 0.00015 and 0.0012 wt. %, preferably between 0.0002 and 0.0006 wt. %, in particular at 0.00028 wt. %, and/or an iron proportion in the first and/or the second preparation lies at 0 wt.% or between 0.015 and 0.09 wt. %, preferably between 0.03 and 0./09 wt. %, in particular at 0.08 wt. %, and/or a vitamin D proportion lies between 0.00001 and 0.00008 wt. %, preferably between 0.00002 and 0.00006 wt. %, in particular at 0.00004 wt. %, and/or a proportion of a catalyst and/or active ingredient for resorption improvement, in particular a piperine proportion, in the first and/or the second preparation lies between 0.0015 and 0.17 wt. %, preferably between 0.005 and 0.034 wt. %, in particular at 0.008 wt. %.

22. The nutritional supplement preparation set according to claim 13, wherein in each of the first and/or the second preparation, the first and/or the second is present in powder form and/or is designated and intended for a production of an aqueous solution and/or is designed and intended for a production of a drink, in particular a mineral-based drink.
